(19) United States
(12) Patent Application Publication
(10) Pub. No.: US 2026/0207480 A1
(43) Pub. Date: Jul. 23, 2026

(54) ESTRIOL VAGINAL RING

(71) Applicant: VaRi Bioscience GmbH, Wildau (DE)

(72) Inventor: Klaus NICKISCH, Wildau (DE)

(21) Appl. No.: 19/134,599

(22) PCT Filed: Dec. 1, 2023
(86) PCT No.: PCT/EP2023/083984

§ 371 Date: May 30, 2025

(30) Foreign Application Priority Data
Dec. 2, 2022 (EP) 22211162.7

(51) Int. Cl. A61K 9/00; A61J 3/00; A61K 31/565; A61K 47/32

(52) U.S. Cl. CPC A61K 9/0036; A61J 3/00; A61K 31/565; A61K 47/32

(57) ABSTRACT

Disclosed is a vaginal ring delivering estriol. The ring is suitable for use in the treatment of vaginal and vulva atrophy. It has a core-shell structure of ethylene vinyl acetate copolymers. Both the core and the shell are loaded with estriol, with the concentration of estriol in the core exceeding that in the shell. The core and shell have a different copolymer composition, with the vinyl acetate in the core being higher than that in the shell.

### FIELD

This disclosure relates to vaginal rings for the sustained delivery of estriol. The disclosure particularly relates to vaginal rings that are suitable for use in the treatment of moderate to severe urogenital symptoms due to postmenopausal atrophy of the vagina (such as dryness, burning, pruritus and dyspareunia) and/or the lower urinary tract (urinary urgency and dysuria), also referred to as vaginal and vulva atrophy (VVA).

### BACKGROUND

Many post-menopausal women are prone to having vaginal and vulva atrophy (VVA), also termed vulvovaginal atrophy or atrophic vaginitis. Other sufferers from VVA include women that are on hormonal anti-cancer therapy, such as breast cancer survivors (BCSs) who initiate adjuvant therapy with nonsteroidal aromatase inhibitors (NSAIs).

The existing treatment of VVA is local estrogen therapy. Mainly, this is provided either by vaginal cream or by pessaries, administering estriol.

A background paper is Buhling et al. Arzneimittelforschung 2012; 62: 378-383. This paper concerns the systemic bioavailability of estriol following single and repeated vaginal administration of 0.03 mg estriol containing pessaries. Such pessaries are available in the market under the name of OEKOLP Ovula 0.03 mg. The regimen of application of Oekolp involves a first stage of 3 weeks during which one pessary is administered daily, and a second stage in which such pessaries are administered twice a week.

As described in the paper, estriol is a naturally occurring short-acting estrogen with a proliferative effect on vaginal, urethral, and bladder epithelium as well as the epithelium of the surface of the cervical os. Local estriol therapy rapidly improves symptoms of estrogen depletion of vagina and lower urinary tract by ameliorating vascularisation and tissue perfusion and promoting proliferation of the vaginal epithelium. Within a few weeks atrophic symptoms like dryness, itching or burning improve significantly. It is believed that the initial administration of estriol will result in thickening of the vaginal epithelium. This has a positive effect on the symptoms of VVA, but it also further reduces the (already low) systemic absorption of estriol. As a result, after the initial phase reducing the symptoms, a second phase, serving to maintain the desired vaginal health, allows a lower exposure to estriol. In the case of Oekolp ovula, this is realized by lengthening the interval between administrations from the initial period (daily) to the maintenance period (twice weekly).

Another background art is Lindén Hirschberg et al., Menopause, Vol. 27, No. 5, 2020. This paper presents the results of a clinical trial on the efficacy and safety of ultra-low dose 0.005% estriol vaginal gel for the treatment of vulvovaginal atrophy in postmenopausal women with early breast cancer treated with nonsteroidal aromatase inhibitors. Here, too, the treatment involves two stages, differing in administration intervals. Using an intravaginal applicator, a dose of 1 g of gel per application (containing 50 mg of estriol) is administered once daily during the first three weeks, and twice weekly during weeks 4 to 12.

Whilst proven and effective, the known treatments of VVA with estriol are not optimal in terms of realizing the aforementioned administration in stages. Changing the administration interval is a known way of realizing different dosing, but it has drawbacks. One issue is patient compliance. For many patients, and increasingly so for the elderly, it is difficult to keep due track of start and stop days of treatment. Also, the administration itself does not involve the desired change in dose. Rather, the same dose is given each time. In the initial stage, this dose is supposed to have a daily effect. In the second stage, however, the very same dosage form is given for the purpose of, in fact, realizing a lower dose over a longer period of time. It will be immediately understood that, without adapting the dosage form itself, the chosen form will by definition not be ideal. For, if the dosage form delivers all of the drug in one day, its potential effect on a stop day, will depend on the body maintaining, or not, a sufficient level of estriol. Even if not working systemically, it cannot be assumed that a dosage given on one day, will still have effect on a next day. Similarly, would a dosage form be adapted release the drug over a period of 3 days' time, the same dosage form would (by definition) not be suitable for use at an initial stage, requiring the same drug dose to be available daily.

For these and other reasons, it would therefore be desired to provide a dosage form for administering estriol that would better ensure the administration of a desired, relatively high, daily dose during an initial phase, yet also of a lower dose, daily available during a next, maintenance phase.

An interesting dosage form to improve patient compliance in the case of VVA, would be an intravaginal dosage form. A well-appreciated example thereof is a drug-loaded intravaginal ring (also known as “vaginal ring”). This specifically relates to polymeric rings, which are easy to insert and which generally are comfortable to keep. These latter factors should possibly support patient compliance, in addition to the sustained release of the drug in lieu of daily intake and, possibly, apart from any actual local effects, the mere logic of treating a vaginal disorder with an intravaginal dosage form.

In the field of vaginal rings, the sustained release of one or more active substances, can be controlled in a variety of ways. This involves a variety of choices for the polymeric material in which the active substances are contained, e.g., silicone rings or rings from polyethylene—polyvinyl acetate. This also involves opting for a matrix system or a reservoir system, opting for one-layered or multi-layered rings, and with or without compartmentalization of the ring.

Reservoir-type vaginal rings generally have a core loaded with an active substance, surrounded by a shell (also indicated as a skin or a membrane) serving the control the release. Examples also exist in which two different active substances are released, with one contained in the core, and the other in the skin.

A general phenomenon occurring with such reservoir-type systems, is the occurrence of the so-called “burst” effect. This refers to a brief, generally uncontrollable, initial release of a peak amount of the active substance, well exceeding the desired actual dose. Such “burst release” can be explained with reference to the amount of active compound that is dissolved in the shell during storage. Here, the active substance is evenly distributed through the shell material at a concentration that is dictated by the concentration existing in the core and the partition coefficient respectively. A freshly produced reservoir will exhibit an empty shell. However the shell will equilibrate with the core within a few days. When the ring is placed, the environment will have a zero concentration of the active substance, and will thus function as a sink, as a result of which the active substance will diffuse out of the shell. In turn, the concentration of the active substance in the shell will decrease until the point where a linear concentration in the shell will be formed. The excess of active substance that is released before the linear gradient is formed is observed as the burst. The end of the burst is defined as the situation at which
a full equilibrium has been settled between core and shell on the one hand, and shell and environment on the other hand. Until then, no steady state is reached, and this results in uncontrolled high release in the beginning.

The aforementioned burst effect, cannot be employed to account for the desired initial release period in the treatment of VVA. For this purpose, the burst release is insufficiently controllable, and occurs for too short a period (typically 1 to 4 days). Moreover, also generally, the burst effect is something that producers and regulatory authorities would wish to avoid. Today, however, regulatory authorities are well aware that the burst effect is expected, more or less inevitably, to occur, and producers will have to provide a justification of how this is treated in their particular situation, with a view to, inter alia, patient safety.

Background art on vaginal rings includes estradiol hormone replacement therapy, and combined estrogen/progestagen contraceptives. U.S. Pat. No. 5,188,835 relates to a single layer siloxane vaginal ring, releasing estradiol. EP876815 discloses a core/shell ring made of ethylene vinyl acetate copolymer, wherein a progestagenic compound and an estrogenic compound are loaded in the core, with the shell being permeable for these compounds. WO 2015/086491 concerns a contraceptive core/shell vaginal ring having a progestagen loaded in the core, and an estrogen loaded in the skin. WO 2019/210134 presents vaginal rings for contraception and hormone replacement. Included is an estriol-releasing ring, which is a matrix system based on a single-layer ring having estriol blended into ethylene vinyl acetate copolymer. None of these disclosures addresses the specific needs in providing VVA therapy.

### SUMMARY

In order to better address one or more of the foregoing desires, we provide, in one aspect, a vaginal ring loaded with estriol as the sole active pharmaceutical ingredient, said vaginal ring comprising a core made of ethylene vinyl acetate random copolymer having a vinyl acetate content in a range of from 20 wt. % to 40 wt. %, said core being surrounded by a shell made of ethylene vinyl acetate random copolymer having a vinyl acetate content in a range of from 4 wt. % to 28 wt. %, wherein both the core and the shell are loaded with estriol.

In another aspect, a method of treatment of VVA is presented, the method comprising administering to a woman in need thereof estriol in a dosage regimen comprising an initial hypertherapeutic dosing and a subsequent maintenance dosing, wherein the estriol is provided by administering a vaginal ring as described in the foregoing paragraph. Similarly, the invention relates to said vaginal ring for use in said method of treatment.

In a still further aspect, the invention provides a method of making a vaginal ring as described above, the method comprising:
a. forming a first mixture of ethylene vinyl acetate random copolymer having a vinyl acetate content in a range of from 20 wt. % to 40 wt. % and estriol, the estriol being in a concentration in a range of from 0.2 wt. % to 5 wt. %;
b. forming a second mixture of ethylene vinyl acetate random copolymer having a vinyl acetate content in a range of from 4 wt. % to 28 wt. % and estriol, the estriol being in a concentration in a range of from 0.01 wt. % to 1.5 wt. %;
c. subjecting the mixtures to hot-melt co-extrusion such that a core-shell extrudate is formed, wherein the first mixture forms a core and the second mixture form a shell encircling the core;
d. allowing the extrudate to cool so as to form a solidified mass;
e. cutting the extrudate, before, during, or after said cooling so as to form one or more cut ring portions corresponding to a desired vaginal ring size, said cut ring portions having two terminal ends;
f. subjecting one or more cut ring portions to bending and putting together said terminal ends so as to form a vaginal ring.

In yet another aspect, the invention provides an intermediate blend comprising estriol blended into ethylene vinyl acetate random copolymer, wherein the copolymer has a vinyl acetate content in a range of from 20 wt. % to 40 wt. % and the estriol is present in a concentration in a range of from 1.25 wt. % to 5 wt. %. Also, the invention provides an intermediate blend comprising estriol blended into ethylene vinyl acetate random copolymer, wherein the copolymer has a vinyl acetate content in a range of from 4 wt. % to 28 wt. % and the estriol is present in a concentration in a range of from 0.01 wt. % to 1 wt. %.

### BRIEF DESCRIPTION OF THE DRAWINGS

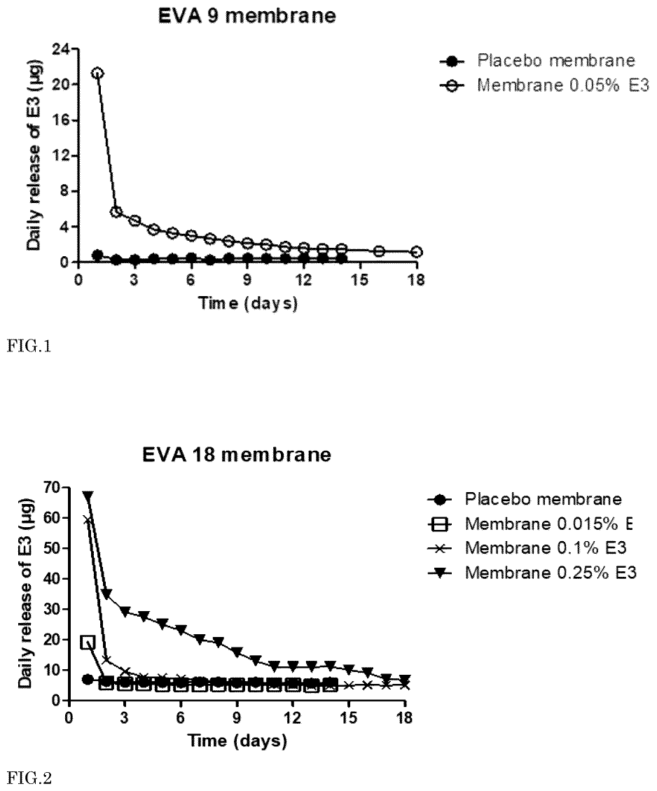

The drawings, i.e., FIG. 1 to FIG. 4, show the daily release of estriol, plotted against time, for various vaginal rings, as discussed in Examples 3 to 5.

### DETAILED DESCRIPTION

The present disclosure relates to an intra-vaginal ring drug delivery system, in short, a vaginal ring. The ring is of the type having a drug-loaded core, functioning as a reservoir, and a release-controlling shell.

Highly surprisingly, the inventors uncovered that the burst effect in the event of the specific combination of estriol as a drug, and ethylene vinyl acetate random copolymer as defined hereinabove, is nearly or entirely absent. The inventors thereby realized that this unexpected phenomenon in fact renders said combination suitable to create a vaginal ring that meets the specific desires for the treatment of VVA.

The invention is thus based on the judicious insight, in deviation from known vaginal rings, to purposely load not only the core with estriol, but also the shell. Without wishing to be bound by theory, the inventors believe that, effectively, the desired initial release is now provided by the shell functioning as a matrix system, whilst after the initial release period the ring will display the characteristics of a reservoir type system. With the present system a much longer initial period of elevated level of release is achievable than in the event of a burst release. Also, the level of the release can be better predetermined than in the event of a mere burst release.

The judicious combination of core and shell polymers is also reflected in the polymer-drug blends that the invention applies in order to produce the rings.

The vaginal ring herein presented, is thus based on the judicious insight to apply the concept of a core/shell vaginal ring to the specific situation of VVA treatment with estriol.

Surprisingly, the present vaginal rings are suitable to satisfy the specific need, in treating VVA, of providing an estriol release profile that
initially provides hypertherapeutic release levels in order to reverse vaginal atrophy and to ensure self-limiting absorption of estriol;
subsequently provides a desired minimal effective dose to sustain the effect on VVA, and avoid systemic absorption of estriol

Without wishing to be bound by theory, the inventors believe that this results from the judicious combination of at least three technical measures. One is to have, despite the delivery being of a single drug only, that same drug also present in the shell. The second is to select the appropriate polymer composition of the shell, in order to effectively deliver the desired initial estriol dose. The third is to select the appropriate polymer composition of the core, in order to effectively deliver the estriol maintenance dose.

In a general sense, the invention is not limited to specific dosage amounts of estriol. For, the same phenomenon underlying the invention, can also be applied to other medical purposes than treating VVA. In fact, the mere absence of a burst release is a benefit that would also apply to rings suitable for estrogenic hormone therapy, or other possible medical applications of estriol-only administration.

In a general sense, the invention is not limited to specific dosage amounts of estriol. For, the same phenomenon underlying the invention, can also be applied for other medical purposes than treating VVA. In fact, the mere absence of a burst release is a benefit that would also apply to rings suitable for estrogenic hormone therapy, or other possible medical applications of estriol-only administration.

In a preferred embodiment, the inventors have found a vaginal ring that is specifically suitable to meet the difficult demands discussed above, for the treatment of VVA. Herein, the aforementioned judicious combination of measures is combined with a fourth measure, viz. to select the appropriate total estriol concentration ranges for core and shell. Hereby, notably, the total concentration in the shell is lower than that in the core. Particularly, the total estriol concentrations are in a range of from 0.2 wt. % to 5 wt. %, such as 1.25 to 5 wt. % in the core, and 0.01 wt. % to 1 wt. % in the shell.

Hereby the total concentration in the core refers to the total weight of estriol contained in the core (whether or not dissolved) relative to the total weight of the core. The total concentration in the shell refers to the total weight of estriol contained in the shell (whether or not dissolved) relative to the total weight of the shell. It will be understood that these total concentrations are determined for the product before it is inserted into the vagina for its intended medical use or, for that matter, before it is put into any aqueous environment that could function as a sink for the estriol loaded in the shell.

It will be understood that the loading of the shell refers to an actual loading when producing the ring. A known phenomenon is that in a core-shell system, over time, diffusion of an active substance active substance to reach an equilibration between shell and core may occur. The vaginal ring of the present disclosure has a shell that is loaded with the specified amount of active substance before such ring is actually put to use by insertion into a vagina, such as when the ring is freshly obtained from its package, such as when the ring is produced and packaged.

Without wishing to be bound by theory, the inventors believe that estriol has a very low solubility in ethylene vinyl acetate (EVA) copolymers. Following conventional logic, this would make the present approach, based on two different EVA grades, an unlikely choice for the skilled person. The inventors have accomplished this by realizing, in marked deviation from conventional single-drug vaginal rings, that the shell can actually assume the function of allowing the release of the initial dose of estriol as needed in the treatment of VVA. It will be understood that in the configuration of the vaginal ring disclosed herein, the release period of the initial dose exceeds that which would normally be denoted as “burst release.”

The present vaginal ring has a core-shell structure. Preferably, the shell refers to a layer that is relatively thin as compared to the core. Accordingly, it is preferred that the diameter of the ring for at least 70% is determined by the diameter of the core, preferably at least 80%, more preferably 90% to 95%. A typical shell layer thickness ranges from 50 μm to 150 μm, such as 80 μm to 120 μm. A most preferred shell layer thickness ranges from 90 μm to 110 μm, such as 95 μm to 105 μm.

The core, in all the aspects of the invention disclosed herein, is selected to be made of ethylene vinyl acetate random copolymer having a vinyl acetate (VA) content in a range of from 20 wt. % to 40 wt. %, such as a VA content of 26 wt. % to 30 wt. %, preferably 28 wt. % (also known as “EVA-28”).

The shell, in all the aspects of the invention disclosed herein, is selected to be made of ethylene vinyl acetate random copolymer having a vinyl acetate (VA) content in a range of from 4 wt. % to 28 wt. %, preferably 4 wt. % to 19 wt. %, more preferably 9 wt. % to 18 wt. %. In an embodiment, the VA content is 8 wt. % to 10 wt. %, preferably 9 wt. % (also known as “EVA-9”). In another embodiment, the VA content is 18 wt. % (“EVA-18”).

As noted above, it is believed that the release mechanisms of core and shell can be considered differently. The core represents a reservoir-type system. Herein the estriol migrates from the reservoir, provided by the core, into the shell. It is thereupon the shell which modulates the release. The shell itself, being loaded with estriol for the initial dose, provides release directly affected by the properties of a polymer matrix (i.e., the EVA composition) and the concentration of estriol in said matrix.

Where it is said that the core and the shell are made of the specified polymers, is will be understood that this does not exclude the presence of adjuvants or excipients in addition to the estriol and the EVA. It will be understood that any additives should meet international pharmaceutical guidelines. These guidelines see, int.al., to requirements for the presence of any leachables and extractables. Since EVA based intrauterine devices such as vaginal rings as such are known and accepted in the art, the skilled person is aware of possible additives. Preferably additives are avoided. A typical additive that, for reasons of preservation, cannot always be avoided in practice concerns antioxidants, such as BHT (butyl hydroxytoluene) or Irganox®. Preferably, the core and the shell consist of the EVA polymer as specified, and the estriol.

Estriol (E3), also spelled oestriol (IUPAC name estra-1,3,5(10)-triene-3,16α,17ß-triol) is a steroid, a weak estrogen, and a minor female sex hormone. It is one of three major endogenous estrogens, the others being estradiol and estrone. Except during pregnancy, estriol plasma levels are low, and thereby hardly detectable. In the course of local estriol therapy of VVA, it has been seen that during a short initial period (e.g. within 12 hours of the first dose intake), estriol plasma levels measurably rise.

Clearly, the goal of the therapy is not to cause a systemic effect (such as is the case with regular peri- and postmenopausal hormone replacement therapy), but to have it act locally. Yet, the initial effect of treatment, as commensurate with the higher initial plasma levels of estriol, is believed to include some systemic effect, resulting in a desirable increase of vaginal epithelium thickness (which, in turn, protects against further systemic uptake of estriol).

The vaginal ring provided by the present invention exceptionally delivers estriol in a manner fitting well with the desired effect. As noted in the art, the corresponding dosing regimen is approached by tuning the corresponding dosage intervals. The vaginal ring of the present invention provides this regimen by the continued releases of estriol, whereby the set-up of the ring is chosen such as to promote such constant release initially being relatively high, whilst thereafter being relatively low.

As said, the invention provides specific estriol concentration ranges for core and shell, thereby notably having a concentration in the shell that is lower than that in the core. The estriol, in all the aspects of the invention disclosed herein, is present in the core at a total concentration in a range of from 0.2 to 5 wt. %, such as from 1.25 wt. % to 5 wt. %, such as from 1.5 wt. % to 2.5 wt. %, such as a weight percentage selected from the group consisting of 1.4 wt. %, 1.5. wt. %, 1.6 wt. %, 1.7 wt. %, 1.8 wt. %, 1.9 wt. %, 2.0 wt. %, 2.1 wt. %, 2.2 wt. %, 2.3 wt. %. and 2.4 wt. %. In an interesting embodiment, the estriol is present in the core at a concentration in a range of from 0.25 wt. % to 1.25 wt. %, such as 0.5 wt. % to 1 wt. %. In a further interesting embodiment, the estriol is present in the core at a concentration in a range of from 1.4 wt. % to 1.6 wt. %. The estriol, in all aspects of this disclosure, is present in the shell at a total concentration in a range of from 0.01 wt. % to 1 wt. %, such as 0.05 wt. % to 0.1 wt. % such as a weight percentage selected from the group consisting of 0.02 wt. %, 0.03 wt. %, 0.04 wt. %, 0.05 wt. %, 0.06 wt. %., 0.07 wt. %, 0.08 wt. %, and 0.09 wt. %; or 0.50 wt. % to 1 wt. %, such as a weight percentage selected from the group consisting of 0.6 wt. %, 0.7 wt. %, 0.8 wt. %, and 0.9 wt. %. In an interesting embodiment, the estriol, in all aspects of this disclosure, is present in the shell at a total concentration in a range of from 0.05 wt. % to 1.5 wt. %.

With knowledge of the present invention, the skilled person will be able to finetune, as desired, the polymer compositions of shell and core, as well as the estriol concentrations therein, within the ranges given.

The present vaginal ring concept allows for a still further improvement over the existing treatment. Initially, the choice of a vaginal ring, providing sustained release of estriol, as such presents a marked improvement over the daily application of cream or gel, or the daily introduction and removal of a pessary. Moreover, however, the present vaginal ring is capable of delivering estriol over a period of three months (more specifically: 90 days). Further, the present vaginal ring allows for a shorter initial dosage period. As indicated above, the existing treatments prescribe that the initial period of more frequent dosing is three weeks. In a preferred embodiment, the vaginal ring of the present invention delivers the initial dose over a period of generally 5 to 20 days, particularly about 1-2 weeks, such as 7-15 days

Another advantage of the present vaginal ring, is that, as compared with the existing treatments, the amounts of estriol involved are substantially lower. E.g., in the event of daily pessaries, each removed pessary will result in residual estriol disposed of. The vaginal rings of the invention will result in a reduced environmental exposure to estriol.

The present vaginal ring may be manufactured by any suitable technique available to the skilled person. Preferably, the core-shell structure of the vaginal ring is produced by means of holt-melt co-extrusion.

The invention thus also relates to a method of making a vaginal ring. The method comprises forming a first mixture of ethylene vinyl acetate random copolymer having a vinyl acetate content in a range of from 20 wt. % to 40 wt. % and estriol, the estriol being in a concentration in a range of from 0.2 wt. % to 5 wt. %. It will be understood, with reference to the present description, that this mixture is presented to form the core of the vaginal ring.

The method further comprises forming a second mixture of ethylene vinyl acetate random copolymer having a vinyl acetate content in a range of from 4 wt. % to 28 wt. %, preferably 4 wt. % tol9 wt. %, more preferably 9 wt. % to 18 wt. %, and estriol, the estriol being in a concentration in a range of from 0.01 wt. % to 1.5 wt. %. It will be understood that this mixture serves to form the shell.

These mixtures can be heated during or after mixing. Heating during mixing can have the advantage of making a more homogeneous blend, taking into account the low solubility of estriol in EVA. The mixture of the EVA and the estriol can be produced using any suitable means. Well-known mixing means known to those skilled in the art include dry mixing, dry granulation, wet granulation, melt granulation, high shear mixing, and low shear mixing.

Alternatively, either or both of the mixtures can be produced by dissolving or dispersing the EVA and the estriol in a suitable solvent, stirring or otherwise homogenizing the solution or dispersion that thereby results, and removing the solvent, such as by evaporation. Suitable solvents include alcohols such as ethanol, acetone, dichloromethane. Also supercritical fluids can be applied.

The two mixtures can either be made by a different method or, as preferred, by the same method. The invention also relates to the blends resulting from the foregoing mixing. These blends, in which the estriol is well-distributed over the EVA, preferably as a homogenous mixture, serve as intermediates to produce the vaginal ring of the invention.

It will be understood that the ring can be made in a direct process involving mixing with heating and followed by co-extrusion of the hot mixtures. The co-extrusion comprises subjecting the mixtures to holt-melt co-extrusion such that a core-shell extrudate is formed, wherein the first mixture forms a core and the second mixture forms a shell encircling the core.

Subsequently the resulting extrudate is allowed to cool so as to form a solidified mass. Such cooling will generally be to a temperature of from 5° C. to 50° C., and typically to room temperature (150 to 25° C., more preferably 18° C.-20° C.).

At some point, the extrudate will be cut in order to provide pieces of extrudate having the desired length for the ultimate vaginal ring. Such cutting can be accomplished before, during, or after the aforementioned cooling. The resulting cut ring portions have two terminal ends, en the length from one end to the other corresponds to a desired vaginal ring size.

Finally, the cut ring portions are subjected to bending and putting together said terminal ends, i.e., ring-shaping so as to form a vaginal ring. The latter can be accomplished by various techniques. In the event that the cut portions are still hot, the ends can be made to meet, and will stick together upon solidification by cooling. Alternatively, different techniques can be used, such as hot air moulding, or by a combination of hot air welding with injection moulding or by using a glue.

The ring can also be made more indirectly, by first making the intermediate blends and later, e.g. after storage and/or transport, subjecting the intermediate blends to heating and hot-melt co-extrusion, followed by the steps of cutting and ring-shaping.

Accordingly, intermediate blends can be provided. One such blend comprises estriol blended into ethylene vinyl acetate random copolymer, wherein the copolymer has a vinyl acetate content in a range of from 20 wt. % to 40 wt. % and the estriol is present in a concentration in a range of from 0.2 wt. % to 5 wt. %. The other intermediate blend comprises estriol blended into ethylene vinyl acetate random copolymer, wherein the copolymer has a vinyl acetate content in a range of from 4 wt. % to 28 wt. %, preferably 4 wt. % to 19 wt. %, more preferably 9 wt. % to 18 wt. %, and the estriol is present in a concentration in a range of from 0.01 wt. % to 1.5 wt. %.

Hot-melt extrusion typically involves the use of an extruder device. Such devices are well-known in the art. Such systems include mechanisms for heating the mixture to an appropriate temperature and forcing the melted feed material under pressure through a die to produce a rod, sheet or other desired shape of constant cross-section.

The vaginal ring according to the invention can be manufactured in any size as required. In practice, however, the ring has an outer diameter of between 50 and 60 mm and more preferably between 52 and 56 mm, the cross-sectional diameter is generally at least 2 mm, preferably more, and at most 10 mm, preferably less. The cross-sectional diameter preferably is between about 2.5 mm and 7.5 mm, more preferably 3 mm to 5 mm. The surface of the core body is preferably more than 800 mm², more preferably at least 1000 mm² and will typically be in the order of 1700-2000 mm². The present vaginal ring is suitable for of a method of treatment of symptoms of vaginal estrogen deficiency in post- and perimenopausal women. Particularly, this refers to moderate to severe urogenital symptoms due to postmenopausal atrophy of the vagina (such as dryness, burning, pruritus and dyspareunia) and/or the lower urinary tract (urinary urgency and dysuria). These symptoms, occurring collectively or individually, are recognized in the field as VVA, i.e., vaginal and vulva atrophy, also referred to as vulvovaginal atrophy.

Provided are, e.g., the following formulations with appropriate release rates:
0.0005 mg/day: core EVA 28+1.5% E3; shell: EVA 9, 0.1 mm, 0.05-0.15% E3; 0.005 mg/day: core EVA 28+1.5% E3; shell: EVA 18, 0.1 mm, 0.8-1.2% E3; 0.025 mg/day: core EVA 28+1,5% E3; shell: EVA 28, 0.1 mm, 0.8-1,2% E3.

In the manufacturing of the vaginal ring described in this disclosure, generally four distinct unit operations can be distinguished:
1) Powder mixing
2) Co-extrusion
3) Cutting
4) Ring closure
1) Pharmaceutical drug products have to meet compendial requirements towards drug content. To achieve the required content uniformity it is necessary to feed the extruders with a homogeneous powder, in which the active ingredient is dispersed in a such a way that within the scale of scrutiny a homogeneous mixture is achieved, yielding the assurance that each single product will have a quantity that is within the specified concentration window. To achieve this, powder should be mixed. This can be done by physically mixing the active pharmaceutical ingredient (API) with the polymer, where the mixing laws give the direction towards the ideal particle sizes to be used; The API should in this respect preferably be micronized and the polymer should exhibit appropriate flowability properties as well as a limited particle size. Polymers are often produced as coarse pellets. Preferably, these are milled to smaller particles of e.g. 300 μm. Alternatively or in addition to the physical mixing, the powder can be mixed with a mixing extruder, which most often (and ideally) is composed of a double screw extruder with dedicated screws, specially designed for the specific application. After cooling, the extrudate is granulated in a strand granulator into particles that are suitable to be fed into the co-extruder equipment.
2) The co-extrusion is performed by an equipment system mostly comprising two single screw extruders which are basically meant to transport and melt the polymers and subsequently built up pressure to the spinneret. Most often and preferably, the extruder is directly connected to a spinning pump, which is a metering pump that is able to dispense an accurate flow to the spinneret in which the membrane is wrapped around the core of the extrudate. In the extruders, adequate temperature ramps are set, yielding the viscosity fit for spinning. The screw design and rotation speed determine the pressure that is offered to the spinning pump. These spinning pumps are selected to exhibit an accuracy of dispensing that is meeting the required specifications of the membrane thickness. By this, full control on the specification towards membrane thickness and hence the anticipated release performance is obtained.
3) The coaxial fibre is cooled e.g. by air or by water and subsequently cut into strands of a defined length.
4) Ring closure involves the connection of the two ends of the strand to a stress and strain resistant joint. This can be achieved by e.g. the application of glue, the use of a mechanical connector, application of a welding technique or the use of mould injection. Preferably the joint does not have a significant influence on the release characteristics nor on the mechanical properties of the final product.

In sum, disclosed is a vaginal ring delivering estriol. The ring is suitable for use in the treatment of vaginal and vulva atrophy. It has a core-shell structure of ethylene vinyl acetate copolymers. Both the core and the shell are loaded with estriol, with the concentration of estriol in the core exceeding that in the shell. The core and shell have a different copolymer composition, with the vinyl acetate in the core being higher than that in the shell.

The invention will hereinafter be illustrated with reference to the following non-limiting examples.

### Example 1 (General Preparation Example)

### Premix Preparation:

Estriol loaded powder blends of ethylene vinyl acetate with vinyl acetate contents between 9 and 33% vinyl acetate and different loadings (in the range of 0.01% to 1.5% w/w) are prepared by dry blending the active agent and the high VA content ethylene vinyl acetate, using different blending techniques (e.g., tumble blending, active blending via high shear forces) and blending parameters, yielding a powder blend where the active agent is homogeneously distributed in the blend.

### Co-Extrusion:

The estriol loaded ethylene vinyl acetate with a higher vinyl acetate content is co-extruded at low throughput ranges of <3 kg/h using a twin screw extruder for the drug loaded core material and a single screw extruder for the estriol loaded ethylene vinyl acetate with lower VA content. E.g. a skin thickness of 100 μm can be achieved in an 18 mm extruder via single screw extruder speeds of <10 rpm. The obtained co-extrudate (reservoir system) is subsequently cooled to yield co-axial fibres with an outer diameter of 4.0 mm and a pre-defined skin thickness. The co-extrudate diameter and sphericity may be controlled in-line using a multiple laser head system.

### Ring Closure:

The estriol loaded reservoir strands are cut into segments of 154 mm either manually or using a semi-automated system prior to being shaped to the vaginal ring via a welding step (e.g., hot air welding, injection moulding) inside a ring-shaped mould with a single or multiple cavities of 4.0 mm cross-sectional diameter and 54 mm outer diameter. As welding material, the drug free ethylene vinyl acetate, serving as the core polymer, is used. The obtained rings are then stored at room temperature.

### Example 2 (General Testing of In Vitro Release Rates)

### Methods

For in vitro dissolution testing, a rotational incubator operated at 37±0.5° C. is used. The type of dissolution medium, its volume and the incubator rotational speed are selected to provide sink conditions. Samples of 1 mL are withdrawn every 24±0.5 h (and multiples thereof) over up to 90 days, the medium is replaced every 24±0.5 h (and multiples thereof) by fresh media and the samples are analysed for the drug content via (ultra) high performance liquid chromatography (UPLC/HPLC).

### Example 3

With reference to the general description of Example 1, vaginal rings #1 and #2 were made on the basis of an EVA28 core and a 100 μm membrane (shell) of EVA9. The E3 concentrations in core and shell are indicated in Table 1.

TABLE 1

| # | Core | Shell |
|---|---|---|
| 1 | 1.5% | 0% |
| 2 | 1.5% | 0.05% |

The E3 release (jig/day) as determined is plotted against time (days 1-18) in FIG. 1. Herein ring #1 is indicated as “Placebo membrane” (black dots) and ring # is indicated as “Membrane 0.05% E3” (white dots). It can be seen that ring #1 exhibits hardly any burst release. It can also be seen that ring #2, while exhibiting a peak release on day 1, presents a period of more than two weeks during which an initial release of E3 is provided that exceeds the remaining steady state reservoir release.

### Example 4

With reference to the general description of Example 1, a series of vaginal rings 3 to 6 was made on the basis of an EVA28 core and a 100 μm membrane (shell) of EVA18. The E3 concentrations in core and shell are indicated in Table 2.

TABLE 2

| # | Core | Shell |
|---|---|---|
| 3 | 1.5% | 0% |
| 4 | 1.5% | 0.015% |
| 5 | 1.5% | 0.1% |
| 6 | 1.5% | 0.25% |

The E3 release (jig/day) as determined is plotted against time (days 1-18) in FIG. 2. In this figure, ring #3 is indicated as “Placebo membrane” (black dots). Rings 4 to 6 are indicated with reference to their respective E3 concentrations in the shell, i.e., ring #4 by “Membrane 0.015% E3”, ring #5 by “Membrane 0.1% E3”, and ring #6 by “Membrane 0.25% E3”.

It can be seen, as with ring #1, that ring #3 exhibits no burst release. Ring #4 shows an initial release on day 1, ring #5 does so for a period of 4 days. It can thus be seen that the initial release period is extended in the event of a higher load of the E3 in the membrane. This confirms, also in conjunction with the absence of a burst release for ring #3, having a placebo (0% E3) membrane, that the release shown cannot be attributed to a burst effect, but rather reflects a higher initial release on day 1, as is normally seen in the event of a matrix system. Finally, ring #6 exhibits an initial release period of 2.5 weeks. From the results thus obtained, the phenomenon underlying the invention is clearly shown. The skilled person will be able to further optimize the polymer compositions and concentrations in order to extend the initial release period.

### Example 5 (Extended Release)

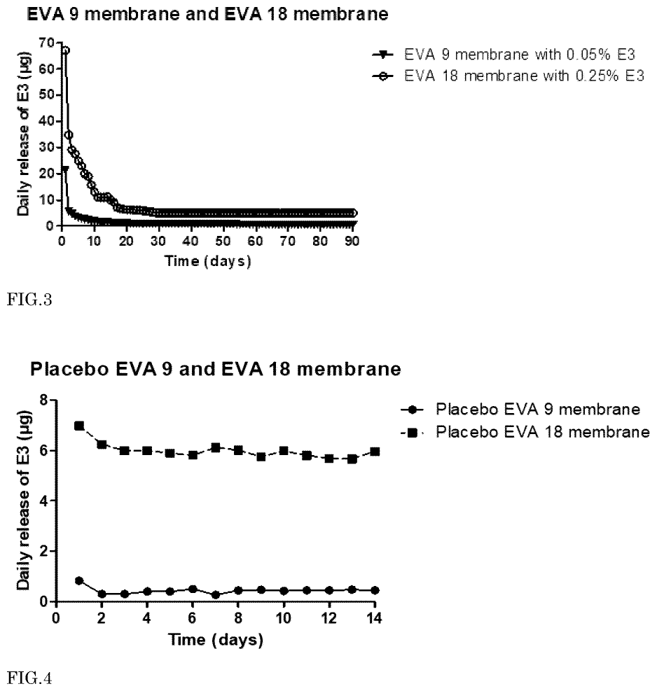

With reference to Examples 3 and 4, an extended period of release was determined for rings #1, #2 and #3, and #6. The results are indicated in FIG. 3 and FIG. 4, both indicating the E3 release (μg/day) plotted against time (days 1-90). Thereby FIG. 3 compares rings #2 (black triangles) and #6 (white dots), whilst FIG. 4 compares rings #1 (black dots) and #3 (black squares).

In FIG. 3 it can be seen that both of the rings exhibit an initial release period of release at a desirably higher level, followed by an extended period of constant release. For ring #2 the initial period is approximately 3 weeks, for ring #6 this is about one month. The constant release level of ring #2 is about 0.5 μg/day, for ring #6 this is about 6 μg/day.

In FIG. 4, it can be seen that ring #1 does not display the typical high release peak as normally seen in the case of a burst effect. Exactly the same pattern is seen with ring #3, with for both rings the constant release level is consistent with that seen in FIG. 3 for the rings having loaded membranes.

1. A vaginal ring loaded with estriol as the sole active pharmaceutical ingredient, said vaginal ring comprising a core made of ethylene vinyl acetate random copolymer having a vinyl acetate content in a range of from 20 wt. % to 40 wt. %, said core being surrounded by a shell made of ethylene vinyl acetate random copolymer having a vinyl acetate content in a range of from 4 wt. % to 28 wt. %, wherein both the core and the shell are loaded with estriol.

2. A vaginal ring according to claim 1, wherein the core is loaded with estriol in a concentration in a range of from 0.2 wt. % to 5 wt. % and wherein the shell is loaded with estriol in a concentration in a range of from 0.01 wt. % to 1 wt. %.

3. A vaginal ring according to claim 1, wherein the core is made of ethylene vinyl acetate random copolymer having a vinyl acetate content of 26 wt. % to 30 wt. % %.

4. A vaginal ring according to claim 1, wherein the shell is made of ethylene vinyl acetate random copolymer having a vinyl acetate content of 8 wt. to 12 wt. %.

5. A vaginal ring according to claim 1, wherein the estriol concentration in the core is 0.2 wt. % to 1.5 wt. %.

6. A vaginal ring according to claim 1, wherein the estriol concentration in the shell is 0.01 to 0.05 wt. %.

7. A vaginal ring according to claim 1, wherein the shell is made of ethylene vinyl acetate random copolymer having a vinyl acetate content of 17-19 wt. %.

8. A vaginal ring according to claim 7, wherein the estriol concentration in the shell is 0.15 to 0.5 wt. %.

9. A vaginal ring according to claim 1, having a shell layer thickness ranging from 80 μm to 120 μm.

10. A vaginal ring according to claim 1, having a cross-sectional diameter in a range of from 2.5 mm to 7.5 mm.

11. A method of treatment of vaginal and vulva atrophy, the method comprising administering to a woman in need thereof estriol in a dosage regimen comprising an initial hypertherapeutic dosing and a subsequent maintenance dosing, wherein the estriol is provided by administering a vaginal ring according to claim 1.

12. A method of treatment of vaginal and vulva atrophy, the method comprising administering to a woman in need thereof estriol in a dosage regimen comprising an initial hypertherapeutic dosing and a subsequent maintenance dosing, wherein the estriol is provided by administering a vaginal ring according to claim 20.

13. A method of making a vaginal ring according to claim 1, the method comprising:
a. forming a first mixture of ethylene vinyl acetate random copolymer having a vinyl acetate content in a range of from 20 wt. % to 40 wt. % and estriol, the estriol being in a concentration in a range of from 0.2 wt. % to 5 wt. %;
b. forming a second mixture of ethylene vinyl acetate random copolymer having a vinyl acetate content in a range of from 4 wt. % to 28 wt. % and estriol, the estriol being in a concentration in a range of from 0.01 wt. % to 1.5 wt. %;
c. subjecting the mixtures to hot-melt co-extrusion such that a core-shell extrudate is formed, wherein the first mixture forms a core and the second mixture forms a shell encircling the core;
d. allowing the extrudate to cool so as to form a solidified mass;
e. cutting the extrudate, before, during, or after said cooling so as to form one or more cut ring portions corresponding to a desired vaginal ring size, said cut ring portions having two terminal ends; and
f. subjecting one or more cut ring portions to bending and putting together said terminal ends so as to form a vaginal ring.

14. A blend comprising estriol blended into ethylene vinyl acetate random copolymer, wherein the copolymer has a vinyl acetate content in a range of from 4 wt. % to 19 wt. % and the estriol is present in a concentration in a range of from 0.01 wt. % to 1.5 wt. %.

15. A blend according to claim 14, wherein the estriol is homogeneously distributed in the copolymer.

16. A vaginal ring according to claim 3, wherein the core is made of ethylene vinyl acetate random copolymer having a vinyl acetate content of 28 wt. %.

17. A vaginal ring according to claim 4, wherein the shell is made of ethylene vinyl acetate random copolymer having a vinyl acetate content of 9 wt. %.

18. A vaginal ring according to claim 9, having a shell layer thickness ranging from 90 μm to 110 μm.

19. A vaginal ring according claim 10, having a cross-sectional diameter in a range of from 3 mm to 5 mm.

20. A vaginal ring loaded with estriol as the sole active pharmaceutical ingredient, said vaginal ring comprising
a core made of ethylene vinyl acetate random copolymer having a vinyl acetate content in a range of from 20 wt. % to 40 wt. %, said core being surrounded by a shell made of ethylene vinyl acetate random copolymer having a vinyl acetate content in a range of from 4 wt. % to 28 wt. %,
wherein both the core and the shell are loaded with estriol,
wherein the core is loaded with estriol in a concentration in a range of from 0.2 wt. % to 5 wt. % and wherein the shell is loaded with estriol in a concentration in a range of from 0.01 wt. % to 1 wt. %,
wherein the shell thickness ranges from 80 μm to 120 μm, and
wherein the ring cross-sectional diameter ranges from 2.5 mm to 7.5 mm.
